(19) United States
(12) Patent Application Publication
(10) Pub. No.: US 2005/0048641 A1
(43) Pub. Date: Mar. 3, 2005

(54) System and method for delivering polynucleotides to the central nervous system

(75) Inventors: Keith R. Hildebrand, Houlton, WI (US); William F. Kaemmerer, Edina, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/962,732

(22) Filed: Oct. 12, 2004

Related U.S. Application Data
(63) Continuation-in-part of application No. 10/721,693, filed on Nov. 25, 2003.
(63) Continuation-in-part of application No. 10/852,997, filed on May 25, 2004.
(60) Provisional application No. 60/429,387, filed on Nov. 26, 2002.

(51) Int. Cl. C12M 1/00

(52) U.S. Cl. 435/283.1

(57) ABSTRACT

Methods and apparatuses for delivering RNA polynucleotides to a patient in need thereof are described. Programmable infusion pump systems that include a reservoir housing the RNA polynucleotide are implanted in the patient. The RNA polynucleotide is delivered to a target location in the patient via a catheter in communication with the reservoir. The pump system may include one or more sensors that may control rate or timing of delivery of the RNA polynucloetide based on a detected event. The pump system allows for controlled delivery of RNA polynucleotides for the treatment of diseases, disorders, or conditions.

### RELATED APPLICATIONS

This application is a Continuation-in-Part of applications Ser. Nos. 10/721,693 and 10/852,997, respectively filed on Nov. 25, 2003 and May 25, 2004, which Applications claims priority to Provisional Applications Serial Nos. 60/429,387 and 60/444,614, respectively filed on Nov. 26, 2002 and Feb. 3, 2003, each of which applications are herein incorporated by reference in their respective entirety.

### FIELD

This disclosure relates to medical devices and methods for delivering polynucleotides to a subject in need thereof.

### BACKGROUND

An important strategy to inhibit the expression of specific genes in mammals is the use of various types of polynucleotides (PNTs), such as antisense oligonucleotides (such as antisense RNA or cDNA), ribozymes, and small interfering RNA (siRNA), in a process generally known as RNA inhibition (RNAi). The ability to translate this method from the lab to the clinic is challenging for several reasons including getting the therapeutic PNT to the desired target tissue, achieving a therapeutic yet safe level of inhibition and maintaining inhibition, i.e. preventing translation of the targeted gene into protein.

One challenge with administering RNAi PNTs for therapeutic purposes involves identifying the appropriate route of administration. Because PNTs are destroyed in the GI tract, they cannot effectively be administered orally (acutely or chronically). Accordingly, the PNTs have been locally delivered to target tissue locations by direct injection. Various techniques for delivering PNTs to cells and target tissues have been used and include encapsulation in liposomes, by iontophoresis, or by incorporation into other vehicles, such as hydrogels, cyclodextrins, biodegradable nanocapsules, and bioadhesive microspheres. Direct injection of PNTs with an appropriate vehicle to a target tissue location overcomes GI degradation, but still presents several problems. Although vascular routes of administration have been used, targeting of the therapy to the organ or tissue of interest has been problematic and delivery to the central nervous system has been especially difficult because of the inability of the PNTs to cross the blood-brain barrier.

For example, the effects of RNAi PNTs are temporary, typically lasting only several days after exposure of the targeted tissue to the PNT. For the purposes of using RNAi PNTs to treat certain diseases, it may be desirable to achieve the effects of RNA inhibition for extended periods of time, up to the life of the patient. As such, strategies to overcome the temporary effect of RNAi PNTs have been developed. One such strategy involves the use of osmotic infusion pumps, such as ALZET® Osmotic Pumps that can continuously deliver the PNT. Osmotic pumps can be attached to a catheter for delivery to a targeted tissue location. While allowing for longer-term delivery of PNTs, osmotic infusion pumps do not allow for a great deal of control over delivery of the PNT. Further, ALZET® Osmotic Pumps are not approved for use in humans, nor are they intended for such use. (see, e.g., http://www.durect.com/wt/durect/page_name/alzet, last visited Sep. 27, 2004).

Other strategies used to circumvent the temporary inhibition associated with PNTs include molecular biological approaches to induce long-term inhibition by using gene therapy to introduce DNA that will encode for and be transcribed into the desired RNAi PNT. There are major risks to such gene therapy approaches. For example, viral vectors that are often used to package the DNA may be immunogenic. Additionally, while not as common with newer vectors, some viral vectors may be virulent or may revert to virulent forms of the virus. Further, once the therapeutic gene is being transcribed to produce the RNAi PNT, it is difficult to turn the process “off”, and many of the consequences of long-term gene silencing (i.e., safety, toxicity) are yet to be appreciated. For example, a protein that is pathogenic in one tissue may be vital in a different tissue. In addition, the level of inhibition may also affect the therapeutic window, i.e., 90% inhibition may be therapeutic whereas 100% inhibition may be toxic if the targeted protein subserves a vital function. To avoid the potential safety issues of viral vectors for gene delivery, various formulations of plasmid DNA have been used although the extent and duration of gene expression has typically not been sufficient to maintain a clinical response.

Currently available and suggested technologies for delivery of RNAi PNTs to a target tissue location, which have been used primarily for pre-clinical purposes, present several obstacles for therapeutic purposes, such as lack of sufficient control or significant risks.

### SUMMARY

Various embodiments of the invention provide systems and methods that allow for chronic silencing of targeted genes without the use of viral vectors, which may be virulent or may revert to pathogenic variants, and various embodiments of the invention provide systems and methods that allow for reversible and controllable infusion of an RNAi PNT into a targeted tissue.

In various embodiments, the invention provides a system comprising an implantable infusion pump device and a fluid housed in a reservoir of the pump device. The fluid comprises an RNAi PNT configured to interact with an RNA molecule of a patient to reduce production of a polypeptide encoded by the RNA molecule. The RNAi PNT may reduce production of the polypeptide by any amount, e.g., by about 100%, by about 90% to about 100%, by about 80% to about 100%, by about 80% to about 90%, by about 70% to about 100%, by about 70% to about 90%, by about 70% to about 80%, by about 60% to about 100%, etc, as compared to production of the polypeptide in the absence of the RNAi PNT. In an embodiment, the RNAi PNT may be configured such that its activity does not exceed about 90%, about 80%, about 70%, about 60%, etc., polypeptide reduction. In an embodiment, the infusion pump device may be programmable so as to deliver differing amounts of the RNAi PNT to a target location within the patient to achieve a desired level of polypeptide reduction. In an embodiment, the system comprises a sensor, capable of detecting an indicator of the level of polypeptide reduction. The sensor may produce a signal capable of altering the amount of RNAi PNT delivered from the pump device based on the level of the indicator detected.

An embodiment of the invention provides a method for reducing expression of a target gene in a patient in need thereof. The method comprises implanting an infusion pump device in the patient. The method further comprises implanting a catheter comprising a proximal end and a delivery portion. The proximal end of the catheter is coupled to the implantable pump and the delivery portion is positioned in a delivery location. The delivery location may be in or adjacent, generally in proximity to, a target tissue to be treated or may be at a distance from the target tissue to be treated. A fluid comprising an RNAi PNT is delivered from the infusion pump device through the catheter to the delivery location. The RNAi PNT may then inhibit the target gene in cells in the target tissue.

In an embodiment, the invention provides a method for treating a disease associated with expression of a gene by inhibiting expression of the gene. The method comprises administering, via an implantable programmable pump, an RNA PNT complementary to a nucleotide associated with the disease.

At least some embodiments of the present invention may provide at least one advantage over currently available or suggested systems and methods for delivering RNAi PNTs. For example, silencing of targeted genes without the use of viral vectors may result in decreased concerns over safety, such as concerns regarding virulence or pathogenicity. In addition, use of a programmable pump allows for titration of RNAi PNT delivery so that an optimal dose range may be obtained and allows for more rapid termination of therapeutic, or non-therapeutic, effects of RNAi PNT therapy. Similarly, delivery systems comprising sensors capable of detecting efficacy, undesired effects, or indicators thereof, and of modulating delivery based on the detection, may allow for titration or termination of RNAi PNT delivery. Further, the use of RNAi PNTs that are configured to produce less than 100% suppression of targeted gene expression may be desirable for treatment of diseases or disorders associated with expression of genes that may also be beneficial to normal cellular, tissue, or system function. Use of RNAi PNTs configured to have reduced half-lives may be useful so that termination of delivery of RNAi PNTs results in quick termination of potentially undesirable effects. While use of RNAi PNTs with reduced half-lives is neither practical nor desired when repeated injections are required, use of such RNAi PNTs with an implantable infusion pump device is practical because the infusion pump system can have a refillable reservoir, allowing for longer-term, less invasive and more controllable delivery.

### BRIEF DESCRIPTION OF THE DRAWINGS

FIG. 1 is a diagrammatic illustration of a system for delivering a composition comprising a therapeutic agent according to an embodiment of the present invention.

FIG. 2 is a diagrammatic illustration of a catheter implanted in a patient according to an embodiment of the present invention.

FIG. 3 is a diagrammatic illustration of a catheter implanted in a patient according to an embodiment of the present invention.

FIG. 4 is a diagrammatic illustration of a system for delivering a composition comprising a therapeutic agent according to an embodiment of the present invention.

The drawings are not necessarily to scale. Like numbers refer to like parts or steps throughout the drawings.

### DETAILED DESCRIPTION

In the following description, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense. Instead, the scope of the present invention is to be defined in accordance with the appended claims.

All scientific and technical terms used in this application have meanings commonly used in the art unless otherwise specified. The definitions provided herein are to facilitate understanding of certain terms used frequently herein and are not meant to limit the scope of the present disclosure.

In the context of the disclosure presented herein, the terms “treat”, “therapy”, and the like are meant to include methods to alleviate, slow the progression, prevent, attenuate, or cure the treated disease.

As used herein, “disease”, “disorder” and “condition” as they refer to the health of a patient are used interchangeably.

As used herein “RNA interfering agent” means any molecule comprising a nucleic acid, or derivative thereof, capable of inhibiting translation of RNA into a polypeptide, and includes antisense oligonucleotides (such as antisense RNA or antisense cDNA), ribozymes, and small interfering RNA (siRNA). An RNA interfering agent “targeted to an RNA of a gene”, “targeted to an mRNA of a gene”, and the like refers to an RNA interfering agent that is complementary to an RNA molecule transcribed from DNA or to an RNA molecule that may be transcribed into a polypeptide.

Delivery System

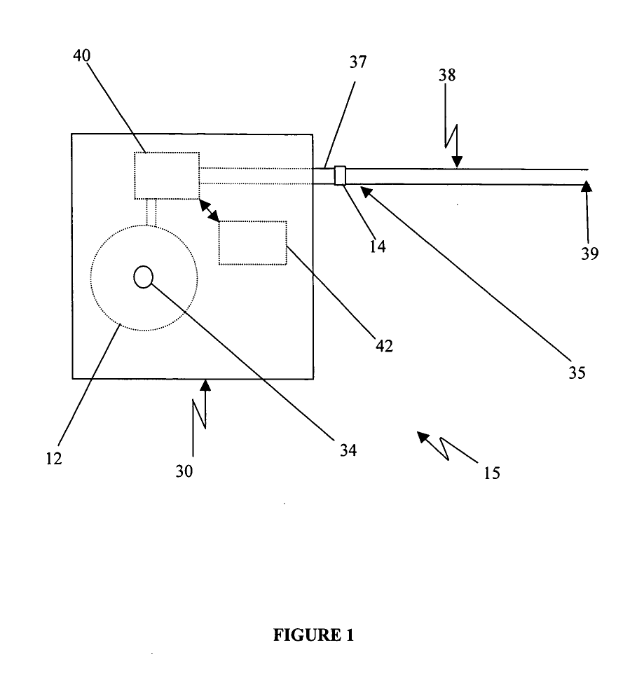

An embodiment of the invention provides a system for delivering to a patient a composition comprising an RNA interfering agent in an amount effective to inhibit translation of an RNA molecule of the patient into a polypeptide or an amount effective to treat a disease. Referring to FIG. 1, a system 15 for delivering a fluid comprising an RNA interfering agent is shown. The system comprises a therapy delivery device 30 or implantable pump device 30. The device 30 comprises a pump 40 coupled to a reservoir 12 for housing a fluid comprising a therapeutic agent, such as an RNA interfering agent. The system 15 further comprises a catheter 38. The catheter 38 comprises a proximal end 35 coupled to the pump 40 and a delivery region 39 adapted for delivering the composition to a delivery location within the patient. It will be recognized that the catheter 38 may have one or more drug delivery regions 39 along the length of the catheter 38 and that a drug delivery region may or may not be at the distal end of the catheter 38. The therapy delivery device 30 may be implantable or may be an external device. The therapy delivery device 30 may have a port 34 into which a hypodermic needle can be inserted to inject a quantity of therapeutic agent into reservoir 12. The device 30 may have a catheter port 37, to which the proximal end 35 of catheter 38 may be coupled. The catheter port 37 is operably coupled to pump 40. A connector 14 may be used to couple the catheter 38 to the catheter port 37 of the device 30. Device 30 may take the form of the device shown in U.S. Pat. No. 4,692,147 (Duggan), assigned to Medtronic, Inc., Minneapolis, Minn., commercially available as the Synchromed® infusion pump, which is incorporated by reference. Device 30 may also take the form of Medtronic's Synchromed® II infusion pump.

The therapeutic device 30, such as Medtronic's SYNCHROMED or SYNCHROMED II pump system, may be operated to discharge a predetermined dosage of the pumped fluid to a delivery location of a patient. Non-limiting delivery locations include the cerebrospinal fluid (CSF) or brain of a patient. The therapeutic device 30 may contain a microprocessor 42 or similar device that can be programmed to control the amount of fluid delivery. The programming may be accomplished with an external programmer/control unit (not shown) via telemetry. A controlled amount of fluid comprising therapeutics may be delivered over a specified time period. With the use of a therapeutic delivery device 30, different dosage regimens may be programmed for a particular patient. Additionally, different therapeutic dosages can be programmed for different combinations of fluid comprising therapeutics. Those skilled in the art will recognize that a programmed therapeutic device 30 allows for starting conservatively with lower doses and adjusting to a more aggressive dosing scheme, if warranted, based on safety and efficacy factors. It will be further recognized that in some situations, it may be desirable to reduce, rather than eliminate, the temporal or spatial expression of a targeted gene in which case a therapeutic delivery device 30 as described herein allows for appropriate dose titration and distribution. Delivery device may also allow for delivery of therapeutic agent to be stopped temporarily and resumed when desired. For example, delivery of agent may be stopped to perform diagnostic tests, intervene with a different therapy, or for safety reasons.

If it is desirable to administer more than one therapeutic agent, the composition within the reservoir 12 may contain a second, third, fourth, etc. therapeutic agent. Alternatively, the therapy delivery device 30 may have more than one reservoir 12 for housing additional compositions comprising a therapeutic agent. When the device 30 has more than one reservoir 12, the pump 40 may draw fluid from the one or more reservoirs 12 and deliver the drawn fluid to the catheter 38. The device 30 may contain a valve coupled to the pump 40 for selecting from which reservoir(s) 12 to draw fluid. Further, one or more catheters 38 may be coupled to the device 30. Each catheter 38 may be adapted for delivering a therapeutic agent from one or more reservoirs 12 of the device 30. A catheter 38 may have more than one lumen. Each lumen may be adapted to deliver a therapeutic agent from one or more reservoirs 12 of the pump 40. It will also be understood that more than one implantable device 30 may be used if it is desirable to deliver more than one therapeutic agent. Such therapy delivery devices, catheters, and systems include those described in, for example, copending application Ser. No. 10/245,963, entitled IMPLANTABLE DRUG DELIVERY SYSTEMS AND METHODS, filed on Dec. 23, 2003, which application is hereby incorporated herein by reference in its entirety.

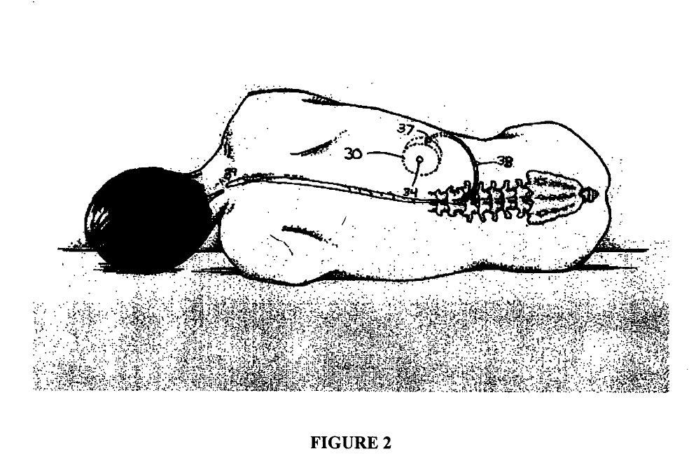

Referring to FIGS. 2, 3, and 4, a system or device 30 may be implanted below the skin of a patient. Preferably, the device 30 is implanted in a location where the implantation interferes as little as practicable with patient activity. Device 30 may be implanted subcutaneously in any medically acceptable area of the human body such as in a subcutaneous pocket located in the chest below the clavicle, in an abdomenal subcutaneous pocket, in the patient's cranium, and the like.

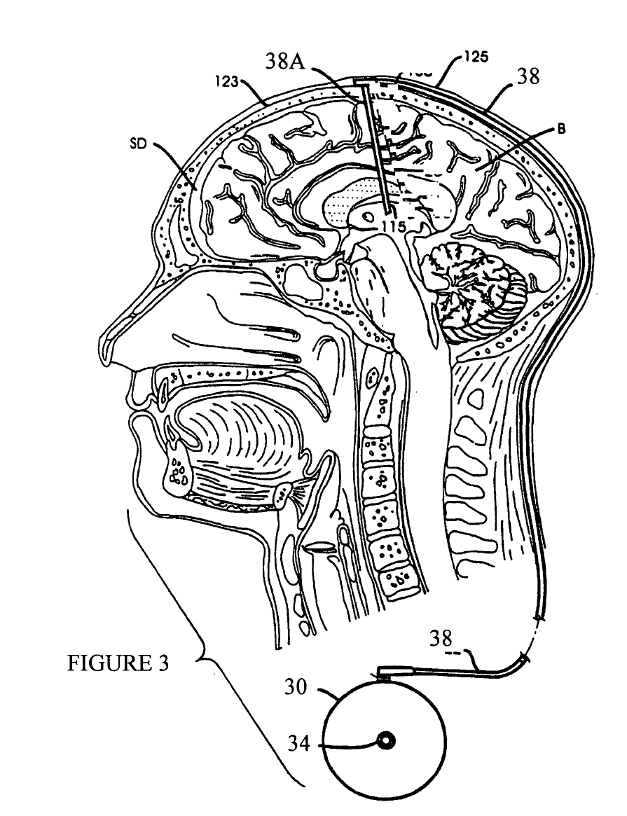

According to an embodiment of the invention, delivery region 39 of catheter 38 is positioned to infuse a fluid into a cerebrospinal fluid (CSF) of the patient. As shown in FIG. 2, catheter 38 may be positioned so that the delivery region 39 (distal tip as shown in FIG. 2) of catheter 38 is located in the subarachnoid space 3 of the spinal cord. It will be understood that the delivery region 39 can be placed in a multitude of locations to deliver a therapeutic agent into the CSF of the patient. The location of delivery region 39 of the catheter 38 may be adjusted to improve therapeutic efficacy. Administering a composition comprising an RNA interfering agent at a level in the spinal canal nearer the brain may result in increased concentrations of an RNA interfering agent in the brain. Decreasing the baricity of a solution or suspension comprising an RNA interfering agent may also result in increased concentrations of the RNA interfering agent in the brain. Alternatively, a composition comprising an RNA interfering agent may be administered directly into the cerebral ventricles. While device 30 is shown in FIG. 3, delivery of a composition comprising an RNA interfering agent into the CSF to treat a CNS disorder or inhibit translation of an RNA in the CNS can be accomplished by injecting the therapeutic agent via port 34 to catheter 38.

Referring to FIG. 3, a system for intraparenchymal or intracerebroventricular (ICV) administration of a fluid comprising an RNAi PNT is shown. Device 30 and delivery system 15 may be as described above. Device 30 and delivery system 15 may take the form of a device and system described in U.S. Pat. No. 6,042,579, entitled “Techniques for treating neurodegenerative disorders by infusion of nerve growth factors into the brain”, which patent is incorporated herein by reference in its entirety. As shown in FIG. 3, the distal end of catheter 38 may terminate in a cylindrical hollow tube 38A having a delivery region 115 (shown in the figure as a distal end) implanted into a portion of the brain by conventional stereotactic surgical techniques. The delivery region 115 may be implanted in the brain in any medically acceptable region. In an embodiment of the invention, the delivery region 115 is implanted in a region within or proximate to cells or tissue having RNA for which inhibition is desired. In an embodiment, delivery region 115 comprises details as described in U.S. application Ser. No. 08/430,960, now abandoned, entitled “Intraparenchymal Infusion Catheter System,” filed Apr. 28, 1995 in the name of Dennis Elsberry et al. and assigned to the same assignee as the present application, which application is herein incorporated by reference in its entirety. Tube 38A may be surgically implanted through a hole in the skull 123 and catheter 38 may be implanted subcutaneously between the skull and the scalp 125 as shown in FIG. 3. Catheter 38 may be joined to implanted device 30 in the manner shown and may be secured to device 30 by, for example, securing catheter 38 to catheter port 37. In an embodiment, delivery region 115 of cylindrical hollow tube 38A may be implanted in a ventricle of the brain. Alternatively, delivery region 115 may be located in the subdural area(SD) beneath the dura under the skull 123 but outside the brain B, and within the subarachnoid space. Catheter 38 may be divided into twin tubes 38A and 38B (not shown) that are implanted into the brain bilaterally. Alternatively, tube 38B (not shown) implanted on the other side of the brain may be supplied with therapeutic agent from a separate catheter 38 and device 30.

While FIGS. 2 and 3 depict administration of a therapeutic agent to a patient's CNS, it will be understood that a therapeutic agent, such as an RNA interfering agent, may be administered to any location within a patient according to various embodiments of the invention. For example, a therapeutic agent may be administered to a patient's kidney or liver or heart, via, e.g., an intra-arterial or intravenous route.

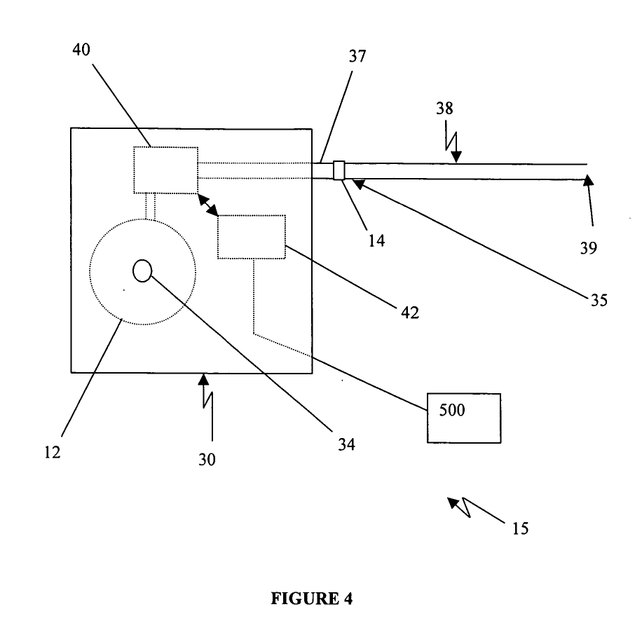

Referring to FIG. 4, the delivery system 15 may include a sensor 500. Sensor 500 may detect an event associated with an effect of the RNA inhibitory agent or the disease to be treated. Sensor 500 may relay information regarding the detected event, in the form of a sensor signal, to processor 42 of device 30. Sensor 500 may be operably coupled to processor 42 in any manner. For example, sensor 500 may be connected to processor via a direct electrical connection, such as through a wire or cable. Sensed information, whether processed or not, may be recoded by device 30 and stored in memory (not shown). The stored sensed memory may be relayed to an external programmer, where a physician may modify one or more parameter associated with the therapy based on the relayed information. Alternatively, based on the sensed information, processor 42 may adjust one or more parameters associated with therapy delivery. For example, processor 42 may adjust the amount and timing of the infusion of PNT. Any sensor 500 capable of detecting an event associated with an effect of the RNA inhibitory agent or the disease to be treated may be used. Preferably, the sensor 500 is implantable. It will be understood that two or more sensors 500 may be employed.

Sensor 500 may detect any event associated with an effect of the RNA inhibitory agent or the disease to be treated may be used. For example, sensor 500 may detect a polypeptide encoded by the target RNA; a product of a enzymatic reaction catalyzed by a polypeptide encoded by the target RNA, such as beta-amyloid as a result of BACE silencing; a physiological effect, such as a change in membrane potential; a clinical response, such as blood pressure; and the like. Any suitable sensor 500 may be used. In an embodiment, a biosensor is used to detect the presence of a polypeptide or other molecule in a patient. Any known or future developed biosensor may be used. The biosensor may have, e.g., an enzyme, an antibody, a receptor, or the like operably coupled to, e.g., a suitable physical transducer capable of converting the biological signal into an electrical signal. In some situations, receptors or enzymes that reversibly bind the molecule being detected may be preferred. In an embodiment, sensor 500 may be a sensor as described in, e.g., U.S. Pat. No. 5,978,702, entitled TECHNIQUES OF TREATING EPILEPSY BY BRAIN STIMULATION AND DRUG INFUSION, which patent is hereby incorporated herein by reference in its entirety, or U.S. patent application Ser. No. 10/826,925, entitled COLLECTING SLEEP QUALITY INFORMATION VIA A MEDICAL DEVICE, filed Apr. 15, 2004, which patent application is hereby incorporated herein by reference in its entirety.

RNA Interfering Agents

Embodiments of the present invention provide systems and methods for delivering RNA interfering agents to a patient in need thereof. Any RNA interfering agent may be used in accordance with the teachings of the present disclosure. Non-limiting examples of RNA interfering agents include various types of PNTs, such as antisense oligonucleotides, such as antisense RNA or cDNA, ribozymes, and small interfering RNA (siRNA). Preferably, the agent is siRNA. A more detailed discussion of siRNA follows. While the following discussion relates primarily to siRNA, it will be understood that many of the concepts presented below are applicable to other PNTs.

As used herein, “small interfering RNA” means a nucleic acid molecule which has complementarily in a substrate binding region to a specified gene target, and which acts to specifically guide enzymes in the host cell to cleave the target RNA. That is, the small interfering RNA by virtue of the specificity of its sequence and its homology to the RNA target, is able to cause cleavage of the RNA strand and thereby inactivate a target RNA molecule because it is no longer able to be transcribed. These complementary regions allow sufficient hybridization of the small interfering RNA to the target RNA and thus permit cleavage. One hundred percent complementarily is often necessary for biological activity and therefore is preferred, but complementarily as low as about 65% may also be useful. The specific small interfering RNA described in the present application are not meant to be limiting and those skilled in the art will recognize that all that is important in a small interfering RNA is that it have a specific substrate binding site which is complementary to one or more of the target nucleic acid regions.

Small interfering RNAs are typically double stranded RNA agents that have complementary to (i.e., able to base-pair with) a portion of the target RNA (generally messenger RNA).

Generally, such complementarily is 100%, but can be less if desired, such as about 90%, about 91%, about 92%, about 93%, about 94%, about 95%, about 96%, about 97%, about 98%, or about 99%. For example, 19 bases out of 21 bases may be base-paired. In some instances, where selection between various allelic variants is desired, a high degree of complementary to the target gene may be desired in order to effectively discern the target sequence from the other allelic sequence. When selecting between allelic targets, choice of length should be taken into account because length is a factor involved in the percent complementary and the ability to differentiate between allelic differences.

The small interfering RNA sequence needs to be of sufficient length to become part of the RNA-induced silencing complex (RISC). The small interfering RNA of the invention may be of varying lengths. The length of the small interfering RNA is preferably greater than or equal to ten nucleotides and of sufficient length to stably interact with the target RNA. In an embodiment, the length of the small interfering RNA is in the range of between about 15 and about 30 nucleotides. Any integer between 15 and 30 nucleotides, such as 15, 16, 30 17, 18, 19, 20, 21, 22, 23, 24, 25, 26, 27, 28, 29, and 30, may be sufficient. By “sufficient length” is meant an oligonucleotide of a length great enough to provide the intended function under the expected condition. By “stably interact” is meant interaction of the small interfering RNA with target nucleic acid (e.g., by forming hydrogen bonds with complementary nucleotides in the target under physiological conditions). In an embodiment, the length of the siRNA sequence is between about 19 and about 30 base pairs. In an embodiment, the length of the siRNA sequence is between about 21 and about 25 base pairs. In an embodiment, the length of the siRNA sequence is between about 21 and about 23 base pairs.

In an embodiment, siRNA is targeted to a complementary sequences in an mRNA sequence coding for production of a target protein, either within the actual protein coding sequence, or in 5′ untranslated region or 3′ untranslated region. After hybridization, host enzymes are capable of cleavage of the mRNA sequence. Perfect or a very high degree of complementarily may be needed for the small interfering RNA to be effective. A percent complementarily indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. It should be noted that single mismatches, or base-substitutions, within the siRNA sequence can substantially reduce the gene silencing activity of a small interfering RNA. However, it may be desirable in some circumstances to reduce the gene silencing activity. For example, when the target protein is required for normal cellular, tissue, organ, or organism function, it may be desirable to reduce, rather than eliminate, production of the protein. In such cases, it is preferred that the siRNA's off-target effects are minimized. Screening of gene arrays may be performed to determine whether off-target effects may be expected for siRNAs with less than perfectly complementary to the target mRNA. Of course any other known or future developed technique may be employed to evaluate potential efficacy of siRNA or potential off-target interactions. In an embodiment, the siRNA is greater than about 80% complementary to the mRNA. In an embodiment, the siRNA is greater than about 90% complementary to the mRNA. In an embodiment, the siRNA is greater than about 95% complementary to the mRNA. In an embodiment, the siRNA is perfectly complementary to the mRNA.

the sequence for a particular therapeutic siRNA can be specified upon knowing (a) the sequence for a small and accessible portion of the target mRNA (available in public human genome databases), and (b) well-known scientific rules for how to identify a sequence that will be an effective siRNA for a given target RNA. See, e.g., Reynolds et al. (2004), Nature Biotechnology 22(3): 326-330, “Rational siRNA silencing design for RNA interference”, which identifies factors that contribute to potency of silencing and an algorithm for manipulating factors to identify potency inhibitors. An siRNA molecule having the identified sequence, once specified, can be constructed using well-known or future developed techniques or purchased from a laboratory supplier.

A brief description of an exemplary method for preparing siRNA follows. siRNAs can be constructed in vitro using DNA oligonucleotides. These oligonucleotides can be constructed to include an 8 base sequence complementary to the 5′ end of the T7 promoter included in the Silencer siRNA kit (Ambion Construction Kit 1620). Each gene specific oligonucleotide may be annealed to a supplied T7 promoter primer, and a fill-in reaction with Klenow fragments generates a full-length DNA template for transcription into RNA.

Two transcribed RNAs (one the antisense of the other) are generated by in vitro transcription reactions then hybridized to each other to make double stranded RNA. The double stranded RNA product may be treated with DNAse (to remove DNA transcription templates) and RNAse (to polish the ends of the double stranded RNA), and column purified to provide the siRNA that can be delivered. For additional details or methods for constructing siRNAs, see, e.g., WO 2004/047872 and the references cited therein. WO 2004/047872 is hereby incorporated herein by reference in its entirety.

To be used with a device 30 or delivery system 15 as described herein, an siRNA molecule may be placed in a composition capable of being pumped through catheter 38. For example, an siRNA molecule may be placed in a fluid composition, such as a solution or suspension. The siRNA may be present in the fluid in any concentration. For example, the siRNA may be present in the fluid at a concentration of between about 0.001 mM and about 100 mM. In an embodiment, the siRNA is present in the fluid at a concentration of between about 0.01 mM and about 1 mM. SiRNA may be administered to a patient at any daily dose effective to treat the disease or disorder at hand. Generally, siRNA will be administered in daily doses of between about 0.1 nmole to about 10 mmole, depending on the stability of the RNA PNT, the location delivered and to be treated, the efficacy of the RNA PNT, and other similar parameters. Generally, it will be desirable to achieve a local tissue concentration of between about 50 pM and about 100 μM of the RNA PNT in the extracellular fluid of the tissue to be treated.

In various embodiments, it may be desirable to modify the siRNA molecule and or the fluid composition comprising the siRNA to affect the stability of the siRNA or the ability of the siRNA to enter cells expressing the target gene. For example, the sugar-phosphate backbone of the siRNA may be modified to enhance stability. U.S. Pat. No. 6,608,036 teaches methods for such modification that may be employed according to the teachings of the present disclosure. In another example, the siRNA may be methylated using known or future developed techniques to affect the stability of the siRNA. The fluid composition comprising the siRNA may further comprise liposomes or cyclodextrin, which may affect both the stability of the siRNA and the ability to penetrate cells. These and other well-known or future developed techniques may be employed to stabilize the siRNA or affect the ability of the siRNA to penetrate cells. In an embodiment, the siRNA is not modified. While unmodified siRNA, once administered, may be too unstable for conventional forms of administration, the use of direct CNS delivery of the siRNA as described herein may allow for the use of less stable RNA due to the proximity of the delivery site to target cells or bypassing the systemic effects. In addition, the use of unmodified siRNA may be advantageous because of its relatively short half-life. For example, because unmodified siRNA will degrade more quickly that some forms of modified siRNA, greater control can be achieved over the ability to stop the effects of unmodified siRNA; i.e., because of the shorter half-life the effects will terminate more rapidly. In an embodiment, the siRNA, whether modified or unmodified, has a half-life between about 1 hour and about 12 hours.

Fluid compositions comprising siRNA include solutions, suspensions, dispersions, and the like. Solutions, suspensions, dispersions, and the like may be formulated according to techniques well-known in the art (see, for example, Remington's Pharmaceutical Sciences, Chapter 43, 14th Ed., Mack Publishing Co., Easton, Pa.) or future developed techniques. If desired, suitable dispersing or wetting and suspending agents, such as sterile oils, including synthetic mono- or diglycerides, and fatty acids, including oleic acid, may be used. Fluid compositions comprising siRNA may be prepared in water, saline, isotonic saline, phosphate-buffered saline, citrate-buffered saline, and the like and may optionally mixed with a nontoxic surfactant. Dispersions may also be prepared in glycerol, liquid polyethylene, glycols, vegetable oils, triacetin, and the like and mixtures thereof. These preparations may contain a preservative to prevent the growth of microorganisms. The prevention of the action of microorganisms can be accomplished by various antibacterial and antifungal agents, for example, parabens, chlorobutanol, phenol, sorbic acid, thimerosal, and the like. In many cases, it will be desirable to include isotonic agents, for example, sugars, buffers, or sodium chloride. Preferably, the fluid composition comprising siRNA is isotonic with the bodily fluid or tissue to which it is delivered. For delivery to CSF, the fluid composition preferably has a tonicity of about 300 mOsm/L. Prolonged absorption of the injectable compositions can be brought about by the inclusion in the composition of agents delaying absorption—for example, aluminum monosterate hydrogels and gelatin. Proper fluidity of solutions, suspensions or dispersions may be maintained, for example, by the formation of liposomes, by the maintenance of the required particle size, in the case of dispersion, or by the use of nontoxic surfactants. In an embodiment, siRNA is dissolved or suspended in water and the fluid composition comprising siRNA is substantially free of preservatives.

Sterile injectable compositions may be prepared by incorporating siRNA in the desired amount in the appropriate solvent with various other ingredients as enumerated above and, as desired, followed by sterilization. Sterile powders comprising siRNA may be used for extemporaneous preparation of sterile injectable or infusible solutions or dispersions. Any means for sterilization may be used. For example, the solution may be filter sterilized. In the case of sterile powders for the preparation of sterile injectable solutions, methods of preparation include vacuum drying and freeze-drying techniques, which yield a powder of the active ingredient plus any additional desired ingredient present in a previously sterile-filtered solution.

Those of skill in the art are familiar with many of the principles and procedures discussed herein and discussed in widely known and available sources, such as Remington's Pharmaceutical Science (17th Ed., Mack Publishing Co., Easton, Pa., 1985); Goodman and Gilman's The Pharmaceutical Basis of Therapeutics (8th Ed., Pergamon Press, Elmsford, N.Y., 1990); Sambrook et al. (1989, Molecular Cloning—A Laboratory Manual, Cold Spring Harbor Laboratories); and Ausubel et al. (1994, Current Protocols in Molecular Biology, Wiley, N.Y.), each of which are incorporated herein by reference in their respective entirety.

Exemplary Therapies/Targets

The systems 15 described herein may be used to deliver RNA PNT therapeutic agents for treating any disease for which RNA PNT therapy may be effective. In an embodiment, the invention provides a method for treating a disease for which reduction of expression of gene may be desired. The method comprises administering to a patient an RNA PNT complimentary to an mRNA transcribed from the gene. The RNA PNT is delivered to the patient via an implantable programmable infusion device 30.

In an embodiment, a method is provided for treating a disease in a patient caused by a dominant gain of function gene mutation. The method comprises administering to a patient, via an implantable programmable pump device 30, an RNA PNT complimentary to an mRNA transcribed from the mutated gene. In circumstances where a dominant gain of function gene mutation is responsible for the disease, it may be desirable to entirely eliminate expression the gene.

In an embodiment, a method is provided for treating a disease in a patient associated with over-expression of a gene that otherwise serves a normal cellular function. The method comprises administering to a patient, via an implantable programmable pump device 30, an RNA PNT complimentary to an mRNA transcribed from the over-expressed gene. In circumstances where over-expression of a normal cellular gene is associated with the disease, it may be desirable to partially reduce expression the gene to a level allowing normal cellular function. The level of reduction desired may be achieved by titrating the amount or timing of RNA PNT delivered from the programmable pump device 30, by varying one or more pump parameter, or by altering the stability or the degree of complementation between the RNA PNT and its polynucleotide target.

In an embodiment, the invention provides a method for treating a CNS disease. The method comprises administering, via an implantable programmable pump device 30, an RNA PNT complementary to a nucleotide associated with the CNS disease. The RNA PNT may be administered intrathecally, ICV or intraparenchymally. The CNS disease may be, e.g., a neurodegenerative disease, a psychiatric disease, epilepsy or cancer.

In an embodiment the invention provides a method for treating amyotrophic lateral sclerosis (ALS). An RNA PNT directed to a mutant form of the Cu, Zn superoxide dismutase (SOD1) gene associated with ALS may be delivered to a patient using a pump system 15 as described herein. The RNA PNT may be, as described in, e.g., Ding et al., Selective silencing by RNAi of a dominant allele that causes amyotrophic lateral sclerosis, Aging Cell 2003; 2: 243-47, which describes selective silencing of ALS alleles without silencing wild-type alleles. The RNA PNT may be delivered directly to the patient's CNS. For example, the RNA PNT may be administered intrathecally, ICV or intraparenchymally. The RNA PNT may be delivered to the motor cortex of the brain or the ventral horn of the spinal cord.

In an embodiment, the invention provides a method for treating cancer, such as a brain tumor. The method comprises administering to a patient, via an implantable programmable pump device 30, an RNA PNT targeted to an oncogene, such as oncogenic K-ras or oncogenic brc/abl, or a multidrug resistance gene, such as MDR1, which encodes P-glycoprotein. The RNA PNT may be delivered in proximity to a tumor. For brain, tumors the RNA PNT may be delivered in proximity to the tumor or into the patient's CSF.

An embodiment of the invention provides a method for treating pain. The method comprises administering, via an implantable programmable pump device 30, an RNA PNT complementary to a nucleotide associated with pain. For example, the RNA PNT may be target to TNF-alpha mRNA, mGlu(1) mRNA, P2X(3) mRNA, or c-fos mRNA. The RNA PNT may be administered intrathecally. An RNA PNT targeted to mGlu(1) may be co-administered with an opioid agonist, such as morphine, as mGlu(1) knockdown can attenuate morphine tolerance.

In an embodiment, the invention provides a method for treating obesity. The method comprises administering, via an implantable programmable pump device 30, an RNA PNT complementary to a nucleotide associated with obesity. For example, the RNA PNT may be targeted to neuro peptide Y (NPY). The RNA PNT may be administered intrathecally, ICV or may be delivered directly to the hypothalamus.

In an embodiment, the invention provides a method for treating allergic encephalomyelitis.

The method comprises administering, via an implantable programmable pump device 30, an RNA PNT complementary to a nucleotide associated with allergic encephalomyelitis. For example, the RNA PNT may be targeted to cytokine responsive gene-2/IP-10. The RNA PNT may be administered intrathecally or ICV.

In an embodiment, the invention provides a method for treating a disease of the liver. The method comprises delivering an RNA PNT targeted to a polynucleotide associated with the disease. The RNA PNT is delivered via an implantable programmable pump device 30 to the patient's liver, e.g., through the hepatic artery or portal vein. The RNA PNT may be complementary to a viral polynucleotide, such as hepatitis a, b, or c, and may be useful for treating viral infections. The RNA PNT may be complementary to an RNA encoding caspase and may be useful for treating liver failure by preventing Fas-mediated apoptosis. The RNA may be complementary to any caspase, such as caspase 8. See, e.g., Zender et al., 2003, “Caspase 8 small interfering RNA prevents acute liver failure in mice”, Proc. Natl. Acad. Sci. USA, 100(13):7797-7802.

In an embodiment, the invention provides a method for treating Parkinson's disease; Alzheimer's disease; Huntington's disease; spinocerebellar ataxia, such as spinocerebellar ataxia type 1, spinocerebellar ataxia type 2, spinocerebellar ataxia type 3, also known as Machado-Joseph disease; or dentatorubral-pallidoloysian atrophy, also known as DRPLA. The method comprises administering, via an implantable programmable pump device 30, an RNA PNT complementary to a nucleotide associated with Parkinson's disease; Alzheimer's disease; Huntington's disease; spinocerebellar ataxia, such as spinocerebellar ataxia type 1, spinocerebellar ataxia type 2, spinocerebellar ataxia type 3, also known as Machado-Joseph disease; or dentatorubral-pallidoloysian atrophy, also known as DRPLA. For example, the RNA PNT may be targeted to alpha-synuclein; beta amyloid cleaving enzyme type 1 (BACE1); mRNA transcript from the IT15 gene, including the code for the huntingtin protein; the mRNA transcript from the SCA1 gene, including the code for the ataxin1 protein; the mRNA transcript from the SCA2 gene, including the code for the ataxin2 protein; the mRNA transcript from the SCA3 gene, including the code for the ataxin3 protein, also known as the Machado-Joseph protein; or mRNA transcript from the DRLPA gene, including the code for the atrophinl protein. The RNA PNT may be administered intrathecally, ICV or may be administered intraparenchymally to, e.g., substantia nigra, nucleus basalis of Meynert; the cerebral cortex; caudate nucleus; putamen; striatum; dentate nucleus; emboliform nucleus; globose nucleus; fastigial nucleus of the cerebellum; cerebellar cortex; or subthalamic nucleus.

An embodiment of the invention provides a method for treating depression. The method comprises administering, via an implantable programmable pump device 30, an RNA PNT complementary to a nucleotide associated with depressive disorders. For example, the RNA PNT may be targeted to corticotropin-releasing factor (CRF), norepinephrine or serotonin reuptake transporters, or substance P receptors. The RNA PNT may be administered intrathecally, ICV or intraparenchymally to, e.g., the hippocampus, amygdala, and the entorhinal cortex. A sensor 500 capable of detecting plasma cortisol may be operably coupled to the device. The sensor 500 may be a chemical sensor or a biosensor. The sensor 500 may be placed in an artery or vein. A chemical sensor could also be placed in the intrathecal space to measure corticotropin levels in the CSF.

An embodiment of the invention provides a method for treating epilepsy. The method comprises administering, via an implantable programmable pump device 30, an RNA PNT complementary to a nucleotide associated with epilepsy. For example, the RNA PNT may be targeted to an enzyme responsible for glutamate production, such as glutamate dehydrogenase; a glutamate receptor, such as an NMDA receptor or an AMPA receptor; a protein that limits the effects of GABA or adensosine, such as degradation enzymes (e.g., GABA-glutamate transaminase or adenosine deaminase) or a GABA or adenosine reuptake transporter. The RNA PNT may be delivered in, at or near, generally in proximity to, an epileptic focus, intrathecally or ICV. A sensor 500 capable of measuring local neural electrical activity may be placed near the focus and may be operably coupled to the device.

1. A system comprising:
an implantable infusion pump;
a reservoir operably coupled to the pump;
a fluid comprising an RNA inhibitory agent, the fluid being housed in the reservoir;
a catheter operably coupled to the pump, the catheter having a delivery region through which the fluid may be delivered; and
a means for controlling the rate at which the fluid is delivered when the pump is implanted in a patient.

2. The system of claim 1, wherein the means for controlling the rate at which the fluid is delivered is a processor.

3. The system of claim 2, wherein the processor is programmable.

4. The system of claim 3, wherein the processor is programmable via telemetry.

5. The system of claim 2, wherein the processor is operably coupled to a sensor.

6. The system of claim 5, wherein the processor alters the rate of fluid delivery based on a signal from the sensor.

7. A system comprising:
an implantable programmable infusion pump;
a reservoir operably coupled to the pump;
a fluid comprising an RNA inhibitory agent, the fluid being housed in the reservoir; and
a catheter operably coupled to the pump, the catheter having a delivery region through which the fluid may be delivered.

8. The system of claim 7, wherein the RNA inhibitory agent is a small interfering RNA (siRNA).

9. The system of claim 8, wherein the siRNA is targeted to a mRNA molecule associated with a disease.

10. The system of claim 9, wherein the siRNA is greater than about 80% complementary to the mRNA.

11. The system of claim 10, wherein the siRNA is greater than about 90% complementary to the mRNA.

12. The system of claim 11, wherein the siRNA is greater than about 95% complementary to the mRNA.

13. The system of claim 12, wherein the siRNA is perfectly complementary to the mRNA.

14. The system of claim 8, wherein the siRNA is targeted to an mRNA of a gene comprising a dominant gain of function mutation.

15. The system of claim 8, wherein the siRNA is targeted to an over-expressed mRNA of a gene that otherwise serves a normal cellular function.

16. The system of claim 8, wherein the siRNA is targeted to an mRNA of a gene associated with a disease of the central nervous system (CNS).

17. The system of claim 8, wherein the CNS disease is selected from the group consisting of a neurodegenerative disease, a psychiatric disease, epilepsy, pain and cancer.

18. The system of claim 8, wherein the siRNA is targeted to an mRNA of a mutant form of Cu, Zn superoxide dismutase (SOD 1) gene associated with ALS.

19. The system of claim 8, wherein the siRNA is targeted to an mRNA of an oncogene.

20. The system of claim 19, wherein the oncogene is oncogenic K-ras or oncogenic brc/abl.

21. The system of claim 8, wherein the siRNA is targeted to an mRNA of a multidrug resistance gene.

22. The system of claim 21, wherein the multidrug resistance gene is MDR1.

23. The system of claim 8, wherein the siRNA is targeted to an mRNA of a TNF-alpha gene, a mGlu(1) gene, P2X(3) gene or a c-fos gene.

24. The system of claim 8, wherein the siRNA is targeted to an mRNA of a neuro peptide Y gene.

25. The system of claim 8, wherein the siRNA is targeted to an mRNA of a cytokine responsive gene-2/IP-10.

26. The system of claim 8, wherein the siRNA is targeted to viral RNA.

27. The system of claim 26, wherein the viral RNA is hepatitis B or hepatitis C RNA.

28. The system of claim 8, wherein the viral RNA is targeted to mRNA of a caspase gene.

29. The system of claim 8, wherein the siRNA is targeted to an mRNA of a gene selected from the group consisting of alpha-synuclein; beta amyloid cleaving enzyme type 1 (BACE1); IT15; SCA1, SCA2, SCA3, and DRLPA.

30. The system of claim 8, wherein the siRNA is targeted to an mRNA of a corticotropin-releasing factor (CRF) gene.

31. The system of claim 30 further comprising:
a sensor operably coupled to the programmable infusion pump, the sensor configured to detect cortisol.

32. The system of claim 8, wherein the siRNA is targeted to an mRNA of a norepinephrine reuptake transporter, a serotonin reuptake transporter, or a substance P receptor.

33. The system of claim 32 further comprising:
a sensor operably coupled to the programmable infusion pump, the sensor configured to detect cortisol.

34. The system of claim 8, wherein the siRNA is targeted to an mRNA of a gene coding an enzyme responsible for glutamate production, a glutamate receptor, a protein that limits the effects of GABA, or a protein that limits the effects of adensosine.

35. The system of claim 34, wherein the siRNA is targeted to an mRNA of a gene coding for glutamate dehydrogenase, an NMDA receptor, an AMPA receptor, GABA-glutamate transaminase, adenosine deaminase, a GABA reuptake transporter, or an adenosine reuptake transporter.

36. The system of claim 34 further comprising:
a sensor operably coupled to the programmable infusion pump, the sensor configured to detect neural activity.

37. The system of claim 35 further comprising:
a sensor operably coupled to the programmable infusion pump, the sensor configured to detect neural activity.

38. The system of claim 7 further comprising a sensor configured to detect a polypeptide translated from an RNA to which the RNA inhibitory agent is targeted.

39. The system of claim 38, wherein the sensor is a biosensor.

40. The system of claim 39, wherein the biosensor comprises an enzyme, an antibody, or a receptor.

41. The system of claim 40, wherein the biosensor comprises an enzyme.

42. The system of claim 41, wherein the biosensor comprises a receptor.

43. A system comprising:
an implantable infusion pump;
a reservoir operably coupled to the pump;
a fluid comprising an RNA inhibitory agent, the fluid being housed in the reservoir, the RNA inhibitory agent being configured to reduce production of a polypeptide by about 25% to about 95%;
a catheter operably coupled to the pump, the catheter having a delivery region through which the fluid may be delivered.

44. A system comprising:
an implantable infusion pump;
a reservoir operably coupled to the pump;
a fluid comprising an RNA inhibitory agent, the fluid being housed in the reservoir, the RNA inhibitory agent being configured to have a half-life of between about 1 hour and about 12 hours when introduced into a delivery location of a patient;
a catheter operably coupled to the pump, the catheter having a delivery region through which the fluid may be delivered to the delivery location.

45. A method for treating a disease associated with expression of a gene, the method comprising:
implanting a programmable pump into a patient, the pump comprising a reservoir housing a fluid comprising an RNA inhibitory agent targeted to an RNA of the gene;
placing a delivery region of a catheter in a delivery region of the patient, the catheter being operably coupled to the pump; and
delivering the fluid through the delivery region of the catheter to the delivery location to allow the RNA inhibitory agent to reduce expression of the gene.

46. The method of claim 45, wherein the delivery location is in proximity to tissue to be treated by the RNA inhibitory agent.

47. The method of claim 45, wherein the delivery location is in the subarachnoid space of the patient.

48. The method of 45, wherein the delivery region is in the patient's brain tissue.

49. The method of claim 45, further comprising sensing an indicator associated with the disease.

50. The method of claim 49, further comprising modifying a delivery parameter of the implantable programmable pump based on information obtained from the sensing.

51. The method of claim 45, further comprising sensing an indicator associated with expression of the gene.

52. The method of claim 51, further comprising modifying a delivery parameter of the implantable programmable pump based on information obtained from the sensing.

53. The method of claim 45, wherein the disease is caused by a dominant gain of function gene mutation.

54. The method of claim 45, wherein the disease is caused by over-expression of a gene that otherwise serves a normal cellular function.

55. The method of claim 45, wherein the disease is CNS disease.

56. The method of claim 55, wherein the CNS disease is selected from the group consisting of a neurodegenerative disease, a psychiatric disease, epilepsy and cancer.

57. The method of claim 55, wherein placing a delivery region of a catheter in a delivery region of the patient comprises placing the delivery region intrathecally, intracerebroventricularly, or intraparenchymally.

58. The method of claim 45, wherein the disease is amyotrophic lateral sclerosis (ALS).

59. The method of claim 58, wherein the gene is a mutant form of the Cu, Zn superoxide dismutase (SOD1) gene associated with ALS.

60. The method of claim 59, wherein placing a delivery region of a catheter in a delivery region of the patient comprises placing the delivery region intrathecally, intracerebroventricularly, or intraparenchymally.

61. The method of claim 59, wherein placing a delivery region of a catheter in a delivery region of the patient comprises placing the delivery region in the patient's motor cortex or the ventral horn of the patient's spinal cord.

62. The method of claim 45, wherein the disease is cancer.

63. The method of claim 62, wherein the gene is an oncogene or a multidrug resistance gene.

64. The method of claim 63, wherein the oncogene is selected from group consisting of oncogenic K-ras and oncogenic brc/abl.

65. The method of claim 63, wherein the multidrug resistance gene is MDR1.

66. The method of claim 62, wherein placing a delivery region of a catheter in a delivery region of the patient comprises placing the delivery region in proximity to a tumor.

67. The method of claim 62, wherein the cancer is brain cancer.

68. The method of claim 67, wherein placing a delivery region of a catheter in a delivery region of the patient comprises placing the delivery region intrathecally, intracerebroventricularly, or intraparenchymally.

69. The method of claim 45, wherein the disease is pain.

70. The method of claim 69, wherein the gene is a gene coding for TNF-alpha, mGlu(1), P2X(3), or c-fos.

71. The method of claim 69, wherein placing a delivery region of a catheter in a delivery region of the patient comprises placing the delivery region intrathecally.

72. The method of claim 45, wherein the disease is obesity.

73. The method of claim 72, wherein the gene is a gene coding for neuropeptide Y.

74. The method of claim 73, wherein placing a delivery region of a catheter in a delivery region of the patient comprises placing the delivery region intrathecally, intracerebroventricularly, or intraparenchymally.

75. The method of claim 73, wherein placing a delivery region of a catheter in a delivery region of the patient comprises placing the delivery region into the patient's hypothalamus.

76. The method of claim 45, wherein the disease is allergic encephalomyelitis.

77. The method of claim 76, wherein the gene is a cytokine responsive gene-2/IP-10.

78. The method of claim 77, wherein placing a delivery region of a catheter in a delivery region of the patient comprises placing the delivery region intrathecally or intracerebroventricularly.

79. The method of claim 45, wherein the disease is a disease caused by a virus.

80. The method of claim 79, wherein the virus is a form of hepatitis.

81. The method of claim 80, wherein the gene is a hepatitis gene.

82. The method of claim 81, wherein placing a delivery region of a catheter in a delivery region of the patient comprises placing the delivery region into the patients portal vein.

83. The method of claim 45, wherein the disease is liver failure.

84. The method of claim 83, wherein the gene is a gene coding for caspase 8.

85. The method of claim 84, wherein placing a delivery region of a catheter in a delivery region of the patient comprises placing the delivery region into the patients portal vein.

86. The method of claim 45, wherein the disease is selected from the group consisting of Parkinson's disease, Alzheimer's disease, Huntington's disease, and spinocerebellar ataxia.

87. The method of claim 86, wherein the gene is selected from the group consisting of alpha-synuclein; beta amyloid cleaving enzyme type 1 (BACE1); IT15, SCA1, SCA2, SCA3, and DRLPA.

88. The method of claim 87, wherein placing a delivery region of a catheter in a delivery region of the patient comprises placing the delivery region intrathecally, intracerebroventricularly, or intraparenchymally.

89. The method of claim 88, wherein placing a delivery region of a catheter in a delivery region of the patient comprises placing the delivery region in the patient's substantia nigra, nucleus basalis of Meynert; the cerebral cortex; caudate nucleus; putamen; striatum; dentate nucleus; emboliform nucleus; globose nucleus; fastigial nucleus of the cerebellum; cerebellar cortex; or subthalamic nucleus.

90. The method of claim 45, wherein the disease is depression.

91. The method of claim 90, wherein the gene is a gene coding for corticotropin-releasing factor.

92. The method of claim 91, wherein placing a delivery region of a catheter in a delivery region of the patient comprises placing the delivery region intrathecally, intracerebroventricularly, or intraparenchymally.

93. The method of claim 92, wherein placing a delivery region of a catheter in a delivery region of the patient comprises placing the delivery region in the patient's hippocampus, amygdala, or entorhinal cortex.

94. The method of claim 90, further comprising sensing cortisol in the patient.

95. The method of claim 94, wherein sensing cortisol comprises sensing cortisol in the patient's blood or cerebral spinal fluid.

96. The method of claim 94, further comprising modifying a delivery parameter of the implantable programmable pump based on information obtained from the sensing.

97. The method of claim 90, wherein the gene is a gene coding for a norepinephrine reuptake transporters, a serotonin reuptake transporter, or a substance P receptor.

98. The method of claim 97, wherein placing a delivery region of a catheter in a delivery region of the patient comprises placing the delivery region intrathecally, intracerebroventricularly, or intraparenchymally.

99. The method of claim 97, wherein placing a delivery region of a catheter in a delivery region of the patient comprises placing the delivery region in the patient's hippocampus, amygdala, or entorhinal cortex.

100. The method of claim 45, wherein the disease is epilepsy.

101. The method of claim 100, wherein the gene is a gene coding for a polypeptide responsible for glutamate production, a glutamate receptor, or a polypeptide that limits the effects of GABA or adensosine.

102. The method of claim 101, wherein the gene is a gene coding for glutamate dehydrogenase, an NMDA receptor, an AMPA receptor, GABA-glutamate transaminase, adenosine deaminase, a GABA reuptake transporter, or adenosine reuptake transporter.

103. The method of claim 100, wherein placing a delivery region of a catheter in a delivery region of the patient comprises placing the delivery region intrathecally, intracerebroventricularly, or intraparenchymally.

104. The method of claim 103, wherein placing a delivery region of a catheter in a delivery region of the patient comprises placing the delivery region in proximity to an epileptic focus.

105. The method of claim 100, further comprising sensing neural activity in a location in proximity to an epileptic focus.

106. The method of claim 105, further comprising modifying a delivery parameter of the implantable programmable pump based on information obtained from the sensing.
